(19) United States
(12) Patent Application Publication
(10) Pub. No.: US 2024/0269450 A1
(43) Pub. Date: Aug. 15, 2024

(54) IMPLANTABLE SKIN-PIERCING DEVICE

(71) Applicant: SECURECELL AG, Urdorf (CH)

(72) Inventor: Daniel Schuetz, Aarwangen (CH)

(21) Appl. No.: 18/271,002

(22) PCT Filed: Jan. 6, 2021
(86) PCT No.: PCT/EP2021/050144

§ 371 Date: Jan. 10, 2024

(51) Int. Cl. A61M 39/02

(52) U.S. Cl. CPC A61M 39/0247; A61M 2039/0202; A61M 2039/0205; A61M 2039/0261; A61M 2039/0264; A61M 2039/0273; A61M 2039/0282

(57) ABSTRACT

An implantable skin piercing device for connecting an external tube to a catheter tube (4) that can be placed inside the human or animal body comprises a cylindrical base body (6) with an upper region (8) facing away from the body in the implanted state and a lower region (10) facing towards the body in the implanted state, wherein the base body is provided in its lower region with a subcutaneous anchorage (12) intended for subcutaneous insertion and wherein upper connection means (16) for connecting the external tube and lower connection means (18) for connecting the catheter tube are present. The subcutaneous anchorage is formed from a plurality of fixing tabs (14) articulated on the base body, which are pivotable from a radially downwards folded retracted position (E) into a radially outwards folded holding position (H). The lower connection means comprise an adapter piece (20) for the catheter tube which can be inserted into the base body and which cooperates with the fixing tabs in such manner that in an inserted lower stop position, it fixes the fixing tabs in the radially outwards folded holding position (H).

### TECHNICAL FIELD

The invention relates to an implantable skin piercing device and a device equipped therewith for supplying and/or withdrawing fluid from a patient.

### PRIOR ART

In patients with a need for repeated or even continuous supply of medication and/or removal of body fluids, appropriate accesses, also called “ports”, are inserted. Such skin piercing devices comprise a port body with an externally accessible port chamber, which is usually connected to a catheter tube located inside the body. The port chamber can be accessed in various ways, in particular by piercing. Subcutaneously implanted ports such as those described in U.S. Pat. No. 5,306,255 A are very common. The corresponding port body lies completely under the skin, and is sealed in the direction of the skin surface by a piercing membrane or by a specially configured piercing unit.

In percutaneously implanted ports, the port chamber is also inserted under the skin, but leaves an entry segment that extends outward through the skin. Such a percutaneous skin piercing device is described in EP 0867197 A2. Compared to subcutaneous devices, there is the advantage of a safe and reliable access without the need for a repeated piercing of a localized skin region.

A generic skin piercing device is described in US 2013/072847 A1.

### DESCRIPTION OF THE INVENTION

The object of the invention is to improve a skin piercing device of the above type and to overcome its disadvantages.

This object is achieved by the skin piercing device defined in claim 1 and by the supply and/or removal device defined in claim 13.

The implantable skin piercing device according to the present invention for connecting an external tube to a catheter tube that can be placed inside the human or animal body, comprises a substantially cylindrical base body with an upper region facing away from the body in the implanted state and a lower region facing the body in the implanted state, wherein the base body is provided in its lower region with a subcutaneous anchorage intended for subcutaneous insertion and wherein upper connection means for connecting the external tube and lower connection means for connecting the catheter tube are available.

Due to the fact that the subcutaneous anchorage is formed from a plurality of fixing tabs articulated on the base body, wherein the fixing tabs are pivotable from a radially downwards folded insertion position into a radially outwards folded holding position, and that the lower connection means comprise an adapter piece for the catheter tube which can be inserted into the base body and which cooperates with the fixing tabs in such manner that in an inserted lower stop position, it locks the fixing tabs in the radially outwards folded holding position, various advantages result.

In particular, the implantation is facilitated due to the initially slim shape in the downwards folded insertion position, whereby, in particular, a comparatively small skin incision is sufficient. Nevertheless, an excellent fixation effect is subsequently ensured because the fixing tabs can act optimally in the locked, radially outwards folded holding position. Moreover, due to the interaction of the adapter piece and the fixing tabs a particularly simple assembly of the device during the course of the implantation process is achieved.

Due to the compact assembly and the mode of action as a percutaneous device, dead volumes during fluid delivery are extremely reduced compared to the conventional subcutaneous devices which require a piercing cup for an external injection needle. The reduction or elimination of dead volumes and the associated mixing or dilution effects, makes it possible to reproduce time-variable fluidic processes with the smallest volumes in a novel way.

In the present context, directional indications are to be understood with regard to the implanted state of the device. Therefore, “facing away from the body” is to be understood in the sense of “facing outwards with respect to the skin surface” and “facing towards the body” correspondingly in the sense of “facing inwards with respect to the skin surface”. For the sake of simplicity, the terms “above” are occasionally used as equivalent to “facing away from the body” and “below” as equivalent to “facing towards the body”, because this corresponds to the graphical representations commonly used in the field. Thus, an “upper region” of the device could actually point downwards, i.e. towards the floor, in certain installation positions and orientations of the patient.

Moreover, geometric specifications are not to be understood in a strictly mathematical sense, but rather in view of the appropriately applicable tolerances.

In addition, it is understood that dimensioning, material selection and material processing are carried out according to the intended purpose of implantation in the human or animal body. Regarding the material for the structural component such as the base body and fixing tabs, there are considered, in particular, titanium, alternatively stainless steel (medical steel) or certain plastics. The dimensions may vary within the usual range. For example, the diameter of the catheter tube will be in the range of 2 to 5 mm depending on application.

Advantageous embodiments are defined in the dependent claims.

The main object of the fixing tabs is to prevent undesired displacements of the implanted skin piercing device. Advantageously (claim 2), the fixing tabs are formed substantially O-shaped and with rounded edges and, in their insertion position, lie within an outline defined by the base body. Due to the central opening, skin can quickly grow into the fixing tab and thus effect the desired function. The embodiment without sharp edges and without protruding structures facilitates the insertion of the skin piercing device by means of a comparatively small skin incision.

In principle, the adapter piece can be held in its lower stop position in various ways. For this purpose, the base body is preferably provided in the lower region thereof with an internal thread, into which a lower threaded ring can be screwed (claim 3). It is particularly advantageous for handling, if the lower threaded ring is provided with a plurality of internal engagement grooves for a correspondingly shaped screwing tool (claim 4).

In one embodiment (claim 5), the base body is provided in its upper region with peripheral depressions for attaching a support ring. This allows to advantageously provide mechanical stabilization during necessary handling such as, in particular, the connecting of the external tube. Undesirable pulling and pushing forces as well as tilting moments are thus avoided.

In an advantageous embodiment (claim 6), the upper connection means comprise a tensioning element which can be screwed into the base body and which comprises a cavity formed to receive a septum body in a filling manner, which cavity is delimited by a lower bottom surface, the latter comprising at least one passage opening.

With regard to the upper connection means, it is advantageous (claim 7), if these comprise a connection head which is connectable to the external tube and which is connectable to the tensioning element. For the required alignment and fixation, the connection head and the tensioning element are provided with cooperating coupling elements. Preferably (claim 8), the cooperating coupling elements are configured according to a plug principle comprising an upper recess in the tensioning element and a corresponding projection in the connecting head.

According to an advantageous embodiment (claim 9), the adapter piece of the lower connection means comprises an adapter head with a base surface and a recess for a sealing insertion of the catheter tube, wherein at least one traversing channel is formed between the recess and the upper base surface. In the assembled state of the skin piercing device, the base surface is directed towards the upper side facing away from the body and forms a contact surface for a tensioning element screwed in above it. Preferably (claim 10), the adapter head is formed in the shape of a dome in such manner that when the adapter piece is inserted into the base body up to the lower stop position, a surface region of the adapter head cooperates with correspondingly shaped end regions of the fixing tabs and pivots the same into the radially outwards folded holding position. This is, therefore, a dome-shaped structure which, in the assembled state of the skin piercing device, is directed to the lower side facing towards the body. Moreover, it is particularly preferred (claim 11) that the recess for the sealing insertion of the catheter tube is arranged laterally on the adapter head. In other words, the catheter tube is guided away substantially parallel to the skin surface below it.

According to a particularly preferred embodiment (claim 12), the traversing channel in the adapter head is formed by at least one, preferably three, cannula(s), wherein each cannula is configured as follows:
a lower cannula end protrudes from the lateral recess,
an adjoining central cannula region is guided in an arc-shaped manner through the adapter head,
an adjoining upper cannula end protrudes from the base surface.

Each laterally protruding lower end of the cannula forms a connection for an associated inner channel of a catheter tube to be attached to the device. Each upper end of the cannula protruding in a direction facing away from the body is intended to reach into an associated septum channel of a septum body attached above it.

In a basic embodiment, the skin piercing device comprises a single fluid channel. For some applications, it is advantageous to have three inner channels, whereby one channel each are used for supplying or withdrawing fluid, while a third channel is provided for receiving a guide wire.

According to a further aspect of the invention (claim 13), a device for supplying and/or withdrawing fluid from a patient, comprises a skin piercing device according to the present invention, a catheter tube connected thereto and an external tube connected thereto, which can be connected to a corresponding process unit. The fluid can, in particular, be a body fluid that has been withdrawn, namely blood from the patient, or an active substance solution that has been supplied. Furthermore, depending on the procedure, it may be necessary at least at certain points in time to convey a rinsing solution through one of the liquid channels or possibly even through both liquid channels.

In this context, it is particularly advantageous (claim 14) if the upper connection means comprise a tensioning element which is screwed into the base body, in the cavity of which a septum body is inserted in a filling manner, wherein the septum body comprises at least one continuous septum channel, into which, on the one hand, an upper cannula end of the adapter piece and on the other hand, a lower tube end of the connection head communicating with the external tube are inserted.

### BRIEF DESCRIPTION OF THE DRAWINGS

Exemplary embodiments of the invention are described in more detail below with reference to the drawings, which show:

FIG. 1 a skin piercing device, in a vertical sectional view;

FIG. 2 the skin piercing device of FIG. 1, in a plan view;

FIG. 3 the skin piercing device of FIG. 1 with attached connection head and external tube, in a vertical sectional view;

FIG. 4 the skin piercing device of FIG. 3, in a plan view;

FIG. 5 the base body of the skin piercing device of FIG. 1, in a vertical sectional view, with the fixing tabs in a radially downwards folded insertion position;

FIG. 6 components of a skin piercing device, in an exploded view;

FIG. 7 a base body with fixing tabs in radially downwards folded insertion position, in a perspective view;

FIG. 8 the base body of FIG. 7, inserted under the skin and with fixing tabs in radially outwards folded holding position, in a vertical sectional view;

FIG. 9 the base body of FIG. 8, with protruding catheter tube, in a vertical sectional view;

FIG. 10 the base body of FIG. 9, with the adapter piece attached to the catheter tube, in a vertical sectional view;

FIG. 11 the base body of FIG. 10, with the adapter piece partially inserted into the base body, in a vertical sectional view;

FIG. 12 the base body of FIG. 11, with the adapter piece completely inserted into the base body and the threaded ring screwed in above it, in a perspective view;

FIG. 13 the base body of FIG. 12, with the adapter piece completely inserted into the base body and the tensioning element screwed in above it, in a perspective view;

FIG. 14 an implanted skin piercing device, in a perspective view;

FIG. 15 the implanted skin piercing device of FIG. 14, with inserted support ring, in a perspective view;

FIG. 16 the implanted skin piercing device of FIG. 15, with attached connection head and external tube, in a perspective view; and

FIG. 17 the implanted skin piercing device of FIG. 16, after removal of the support ring, in a perspective view.

### MODES FOR CARRYING OUT THE INVENTION

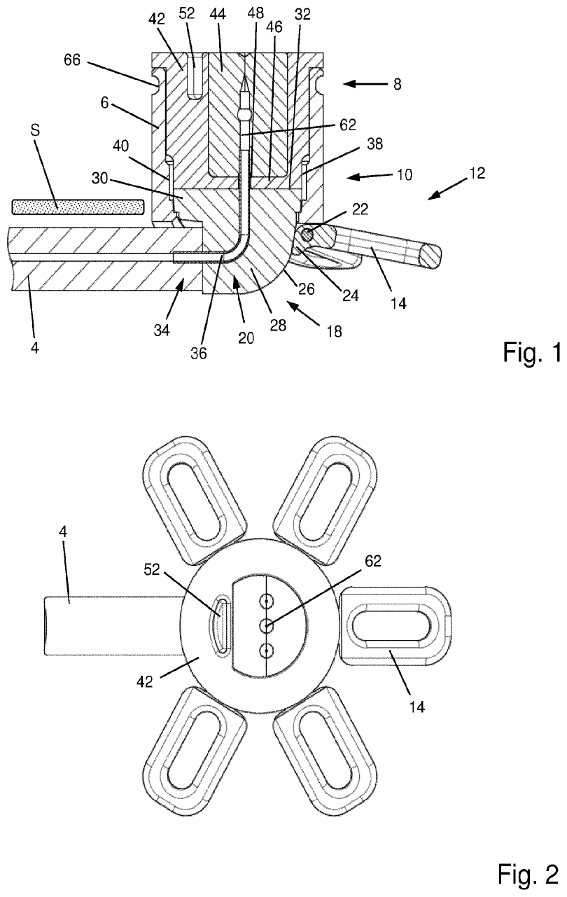

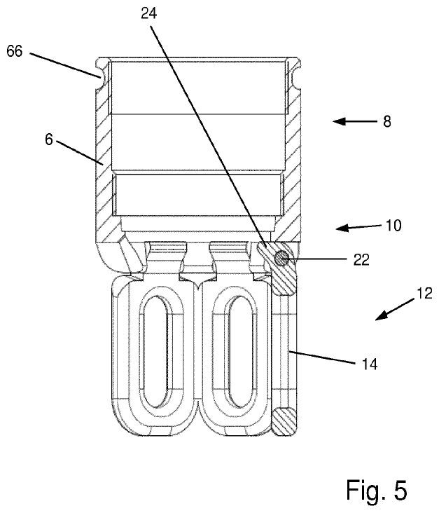

FIGS. 1 to 5 shows an implantable skin piercing device for connecting an external tube 2 to a catheter tube 4 that can be placed inside the human or animal body. The device comprises a substantially cylindrical base body 6 with an upper region 8 facing away from the body in the implanted state and a lower region 10 facing towards the body in the implanted state. To illustrate the installation position in relation to the adjacent skin layer S, a small skin segment is shown in FIG. 1. The base body is provided in its lower region with a subcutaneous anchorage 12 intended for subcutaneous insertion, which is formed from a plurality of fixing tabs 14 articulated on the base body. The fixing tabs are pivotable from a radially downwards folded insertion position E shown here in FIG. 5 into the radially outwards folded holding position H shown in FIGS. 1 to 4.

Moreover, the skin piercing device is provided with upper connection means 16 for connecting the external tube and lower connection means 18 for connecting the catheter tube.

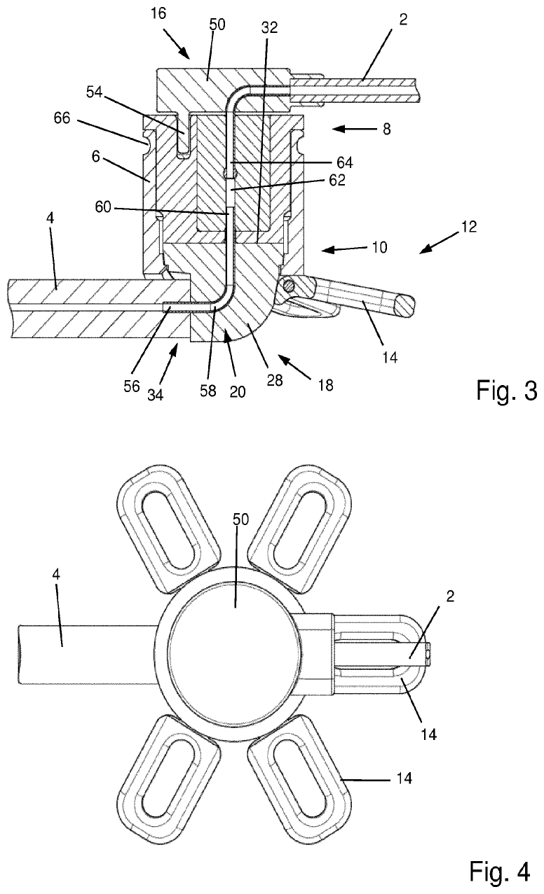

The lower connection means comprise an adapter piece 20 for the catheter tube which can be inserted into the base body and which cooperates with the fixing tabs in such manner that in an inserted lower stop position, shown in FIGS. 1 and 3, it fixes the fixing tabs in the radially outwards folded holding position H. For this purpose, in the example shown, each fixing tab 14 is mounted on an associated pivot axis 22, and has a cam-like end region 24 facing away from the main segment of the tab. The latter rests against a correspondingly shaped outer surface region 26 of a dome-shaped adapter head 28. Thus, by lowering the adapter 20, the end region 24 is also lowered and the main segment of the fixing tab is correspondingly raised and locked. As can also be seen from FIGS. 1 and 3, the adapter head is provided with a flange-like portion 30 with a base surface 32 and a laterally arranged recess 34 for a sealing insertion of the catheter tube, wherein at least one traversing channel is formed between the recess and the upper base surface.

In the example shown, the base body 6 is provided in the lower region thereof with an internal thread 38, into which a lower threaded ring 40 can be screwed in order to hold the adapter piece in its lower stop position.

The upper connection means comprise a tensioning element 42 which can be screwed into the base body and which comprises a cavity formed to receive a septum body 44 in a filling manner, which cavity is delimited by a lower bottom surface 46 with at least one passage opening 48. As can be seen in particular from FIGS. 3 and 4, the upper connection means comprise a connection head 50 which is connectable to the external tube and which is connectable to the tensioning element on the upper side, wherein the connection head and the tensioning element are provided with cooperating coupling elements. In the example shown, these are in each case an upper recess 52 in the tensioning element and a corresponding projection 54.

As shown particularly in FIGS. 1 to 3, the traversing channel in the adapter head 28 is formed by three cannulas, wherein each cannula is configured as follows:
a lower cannula end 56 protrudes from the lateral recess 34,
an adjoining central cannula region 58 is guided in an arc-shaped manner through the adapter head,
an adjoining upper cannula end 60 protrudes from the base surface and is preferably provided with a point. This upper end of the cannula leads into a traversing channel 62 of a septum body 44 attached above it.

In FIGS. 3 and 4, the skin piercing device is shown in a ready-to-use state, wherein a lower tube end 64 of the connection head 50 communicating with the external tube 2 is inserted into the traversing channel 62 of septum body 44.

From a synopsis of FIGS. 1 and 3 it is evident, that the septum 44 comprises an inner bore 62, which is pierced by the cannula 64 and thus expanded.

As can be seen from FIGS. 2 and 4, the fixing tabs are formed substantially O-shaped and with rounded edges. Furthermore, the base body is provided in its upper region with peripheral depressions for attaching a support ring.

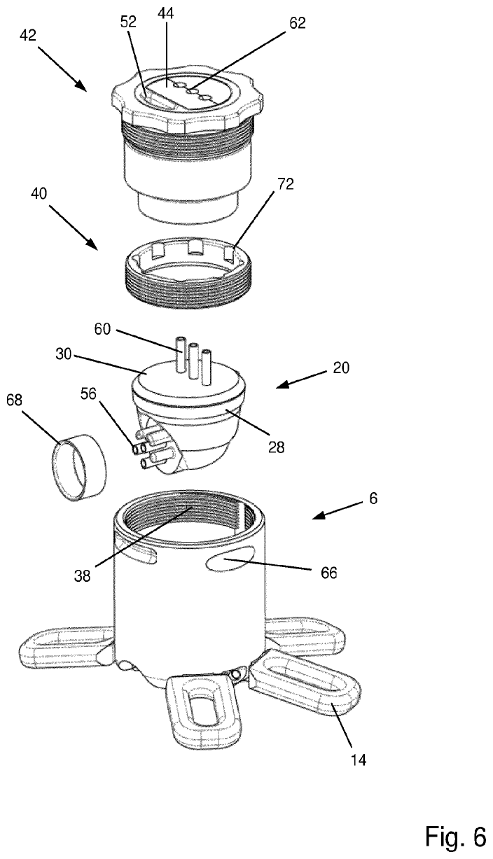

The operating principle and handling of the skin piercing device are further explained in FIGS. 6 to 17. Features already introduced with the above figures are provided with corresponding reference numbers and are mentioned again only where necessary.

FIG. 6 first shows important components of a skin piercing device, in a exploded view. In addition to the components already discussed, there is shown a clamping ring 68 for the catheter tube.

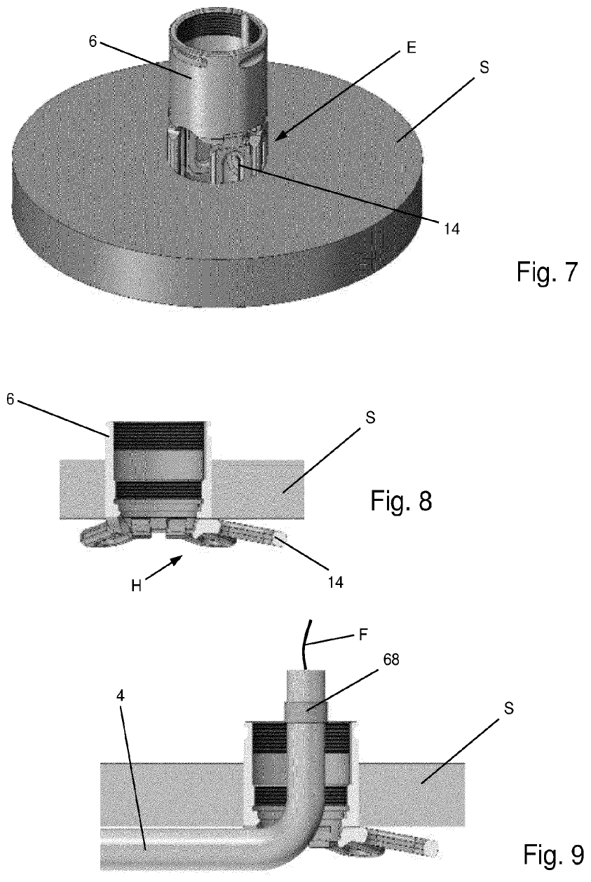

FIG. 7 shows a base body 6 with fixing tabs 14 in radially downwards folded insertion position, above of a schematically shown skin region H. It can be seen that the fixing tabs in their insertion position lie within an outline defined by the base body. Accordingly, a comparatively small segment has to be cut open in the skin, which would not be sufficient with a conventional skin piercing device with rigid subcutaneous anchorage. FIG. 8 then shows the situation with fixing tabs in the radially outwards folded holding position.

In the practical surgical procedure, before the insertion of the skin piercing device, the catheter tube 4 is inserted in basically known manner and inserted at the desired location, for example into a blood vessel. This is usually done with the aid of a guide wire F. This is illustrated in FIG. 9, where the clamping ring 68 placed on the catheter tube can also be seen.

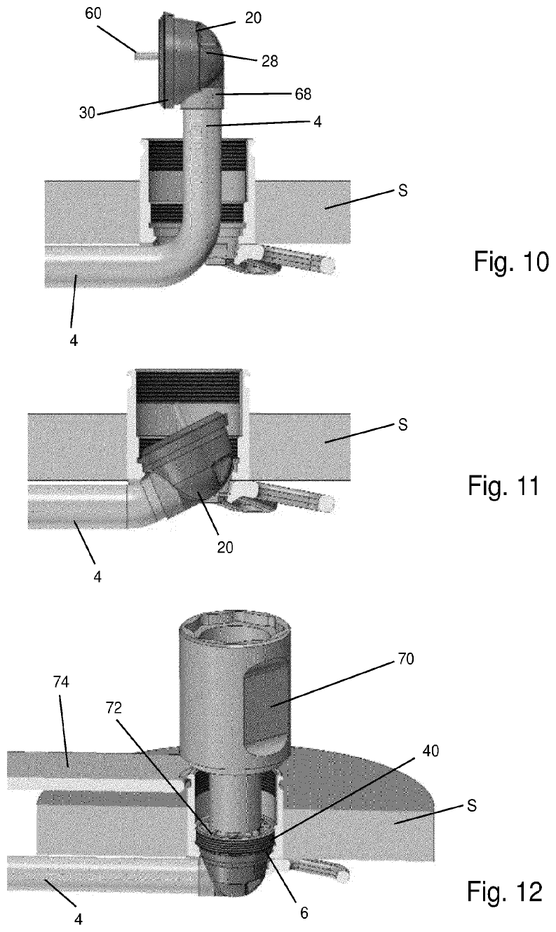

FIG. 10 shows the situation after an adapter piece 20 has been attached to the catheter tube and fixed by clamping the clamping ring 28. Subsequently, the catheter tube 4 was drawn into the body, which is done in known manner via a loop guide to a second skin incision. This leads to the situation shown in FIG. 11, in which the adapter piece is pulled into the basic body.

FIG. 12 then shows the base body of FIG. 11 with completely inserted adapter piece 20 and the threaded ring 40 screwed in above it. Also shown is a screwing tool 70 which is inserted with a first end into correspondingly configured engagement grooves 72 of the threaded ring. Moreover, a holding tool 74 is shown, which grips the base body 6 at the already mentioned peripheral depressions 66.

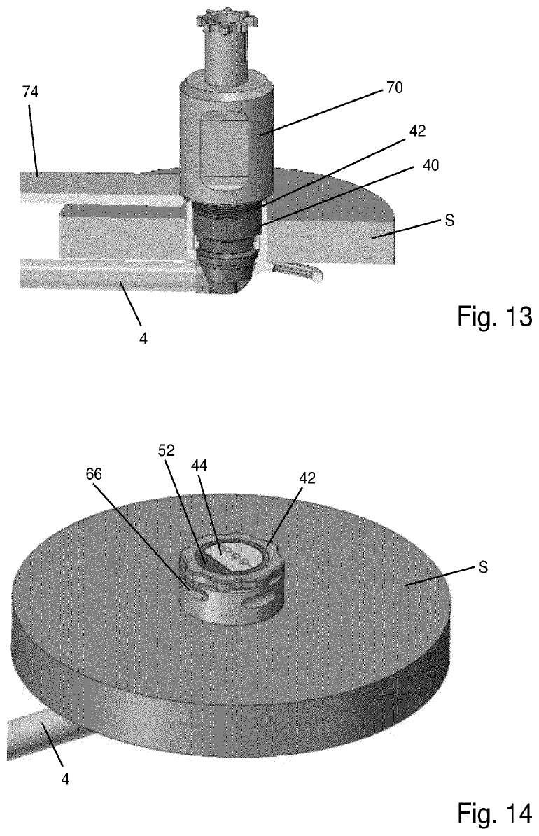

FIG. 13 shows the situation with adapter piece 20 completely inserted into the base body and the tensioning element 42 screwed in above it. For this purpose, the screwing tool 70 is again advantageously used, which is configured at its second end like a ring wrench and thus grips a correspondingly shaped upper edge zone of the clamping element. After removing the screwing tool and the retaining tool, the situation shown in FIG. 14 finally results.

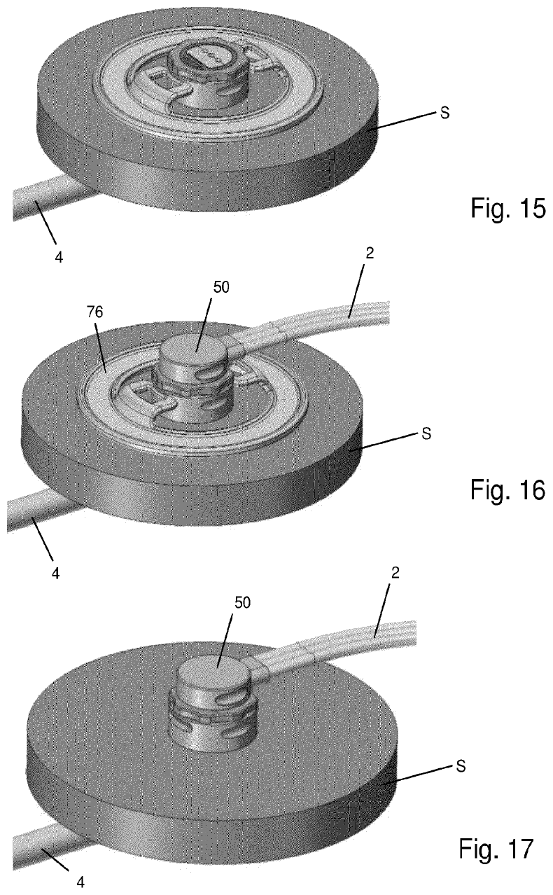

FIGS. 15 to 17 show the attachment of the connection head 50 connected to the external tube 2. First, a specifically configured support ring 76 is attached to the aforementioned peripheral depressions 66 of the base body 6. Subsequently, the connection head 50 is put on and finally the support ring 76 is removed again. The skin piercing device is thus basically ready for operation.

The skin piercing device described above by way of example can be connected in a fundamentally known manner to a corresponding process unit, not shown here, for supplying and/or withdrawing fluid from a patient.

1. An implantable skin piercing device for connecting an external tube to a catheter tube that can be placed inside a human or animal body, comprising:
a substantially cylindrical base body with an upper region facing away from the body in an implanted state and
a lower region facing the body in the implanted state, wherein the base body is provided in the lower region with a subcutaneous anchorage configured for subcutaneous insertion and
wherein upper connection means for connecting the external tube and lower connection means for connecting the catheter tube are provided,
wherein
the subcutaneous anchorage is formed from a plurality of fixing tabs pivotably linked to the base body, and wherein the fixing tabs are pivotable from a radially downwards folded insertion position into a radially outwards folded holding position, and
wherein the lower connection means comprise an adapter piece for the catheter tube which can be inserted into the base body and which cooperates with the fixing tabs in such manner that in an inserted lower stop position, it fixes the fixing tabs in the radially outwards folded holding position.

2. The skin piercing device according to claim 1, wherein the fixing tabs are substantially O-shaped and with rounded edges and, in the radially downwards folded insertion position, lie within an outline defined by the base body.

3. The skin piercing device according to claim 1, wherein the base body is provided in the lower region with an internal thread, configured to have a lower threaded ring can be screwed into it in order to hold the adapter piece in the lower stop position.

4. The skin piercing device according to claim 3, wherein the lower threaded ring is present to hold the adapter piece in the lower stop position and is provided with a plurality of internal engagement grooves for a correspondingly shaped screwing tool.

5. The skin piercing device according to claim 1, wherein the base body is, in the upper region, provided with peripheral depressions for attaching a support ring or holding tool.

6. The skin piercing device according to claim 1, wherein the upper connection means comprise a tensioning element which is configured to be screwed into the base body and which comprises a cavity formed to receive a septum body in a filling manner, which cavity is delimited by a lower bottom surface with at least one passage opening.

7. The skin piercing device according to claim 6, wherein the upper connection means further comprises a connection head which is configured to be connected to the external tube and which is further configured to be connected to the tensioning element on the upper side, wherein the connection head and the tensioning element are provided with cooperating coupling elements.

8. The skin piercing device according to claim 7, wherein the cooperating coupling elements comprise an upper recess in the tensioning element and a corresponding projection in the connecting head.

9. The skin piercing device according to claim 1, wherein the adapter piece of the lower connection means comprises an adapter head with a base surface and a recess for a sealing insertion of the catheter tube, wherein at least one traversing channel is formed between the recess and an upper part of the base surface.

10. The skin piercing device according to claim 9, wherein the adapter head is formed in the shape of a dome in such manner that when the adapter piece is inserted into the base body up to the lower stop position, a surface region of the adapter head cooperates with correspondingly shaped end regions of the fixing tabs and pivots these into the radially outwards folded holding position.

11. The skin piercing device according to claim 10, wherein the recess for a sealing insertion of the catheter tube is arranged laterally on the adapter head.

12. The skin piercing device according to claim 11, wherein the traversing channel in the adapter head is formed by at least one cannula, wherein each cannula is configured as follows:
a lower cannula end protrudes from the lateral recess,
an adjoining central cannula region is guided in an arc-shaped manner through the adapter head,
an adjoining upper cannula end protrudes from the base surface.

13. A device for supplying and/or withdrawing fluid from a patient, comprising: a skin piercing device according to claim 1, the catheter tube connected thereto and the external tube connected thereto, which is configured to can be connected to a corresponding process unit.

14. The device according to claim 13, wherein the upper connection means comprise a tensioning element which is screwed into the base body, in the cavity of which a septum body is inserted in a filling manner, wherein the septum body comprises at least one continuous septum channel, into which, an upper cannula end of the adapter piece and a lower tube end of the connection head communicating with the external tube are inserted.

15. The skin piercing device according to claim 2, wherein the base body is provided in the lower region with an internal thread, configured to have a lower threaded ring be screwed into it in order to hold the adapter piece in the lower stop position.

16. The skin piercing device according to claim 15, wherein the lower threaded ring is present to hold the adapter piece in the lower stop position and is provided with a plurality of internal engagement grooves for a correspondingly shaped screwing tool.

17. The skin piercing device according to claim 16, wherein the base body is, in the upper region, provided with peripheral depressions for attaching a support ring or holding tool.

18. The skin piercing device according to claim 5, wherein the upper connection means comprise a tensioning element which is configured to be screwed into the base body and which comprises a cavity formed to receive a septum body in a filling manner, which cavity is delimited by a lower bottom surface with at least one passage opening.

19. The skin piercing device according to claim 18, wherein the upper connection means further comprises a connection head which is configured to be connected to the external tube and which is further configured to be connected to the tensioning element on the upper side, wherein the connection head and the tensioning element are provided with cooperating coupling elements.

20. The skin piercing device according to claim 12, wherein the traversing channel in the adapter head is formed by at least three cannulas.
